(19) United States
(12) Patent Application Publication
(10) Pub. No.: US 2026/0098071 A1
(43) Pub. Date: Apr. 9, 2026

(54) GLP-1 Analog and Use Thereof in Treatment of GLP-1 Related Diseases

(71) Applicant: Tao Sun, Xiamen (CN)

(72) Inventor: Tao Sun, Xiamen (CN)

(21) Appl. No.: 18/968,327

(22) Filed: Dec. 4, 2024

(30) Foreign Application Priority Data
Oct. 9, 2024 (CN) 202411401343.9

(51) Int. Cl. C07K 14/575; A61K 38/00; A61P 3/04; A61P 3/10; A61P 25/22; A61P 25/24

(52) U.S. Cl. CPC C07K 14/575; A61P 3/04; A61P 3/10; A61P 25/22; A61P 25/24; A61K 38/00

(57) ABSTRACT

The present disclosure relates to a novel human glucagon-like peptide-1 (GLP-1) analog. Compared with the natural sequence of GLP-1, the GLP-1 analog has a remarkable effect in improving GLP-1-related diseases such as obesity, type 2 diabetes mellitus (T2DM), and fatty liver (FL), as well as mood disorders such as anxiety and depression.

### CROSS-REFERENCE TO RELATED APPLICATION

This application claims priority to Chinese Patent Application No. 202411401343.9, filed Oct. 9, 2024, the disclosure of which is hereby incorporated by reference in its entirety.

### REFERENCE TO A SEQUENCE LISTING

The Sequence Listing associated with this application is filed in electronic format via Patent Center and is hereby incorporated by reference into the specification in its entirety. The name of the file containing the Sequence Listing is 2408395.xml. The size of the file is 5,260 bytes, and the file was created on Nov. 18, 2024.

### BACKGROUND

### Technical Field

The present disclosure relates to the technical field of biomedicines, and in particular, to a GLP-1 analog and use thereof in treatment of GLP-1 related diseases.

### TECHNICAL CONSIDERATIONS

Glucagon-like peptide-1 (GLP-1) is a peptide hormone that is cleaved from proglucagon, and contains 31 amino acids. GLP-1 is mainly secreted by L cells in the distal intestine, cells in the pancreas and the central nervous system. GLP-1 is widely distributed in various parts of the human body and is associated with abnormal symptoms in humans. The half-life of GLP-1 in the blood is approximately 2 minutes, and only 10%-15% of GLP-1 enters the circulation. GLP-1 is mainly degraded by dipeptidyl peptidase-4 (DPP-4) and neutral endopeptidase (NEP) in the body and loses its activity [1]. The effects of GLP-1 include suppressing appetite, delaying gastric emptying, inducing weight loss, regulating inflammation in the body, and improving related diseases of the metabolic system, cardiovascular system, nervous system, and digestive system [2]. Obesity, diabetes mellitus and related diseases are the areas of high concern directly related to GLP-1.

The development of obesity increases the risk of suffering from diseases such as diabetes mellitus (DM) and fatty liver (FL) [3]. Obesity refers to the fat mass stored in the body exceeding 20% of ideal body weight, and is caused by the interaction of genetics, environment, individual habits and other factors, making energy intake exceed consumption, thus leading to excessive accumulation of fat in the body and excessive body weight. DM is a chronic metabolic disease characterized by insufficient insulin secretion from pancreatic islet B-cells and blood glucose rise caused by tissue insulin resistance. Obesity, sedentary life, high-calorie diets and increase of an aging population are the main driving factors of DM. According to data disclosed by the International Diabetes Federation (IDF), patients with DM have a death risk increased by 15% [4]. FL is a disease characterized by pathological changes in the liver. FL is formed when liver cells contain triglycerides exceeding 5%. Studies have shown that unhealthy diet and lack of physical activities are determining factors of FL [5].

GLP-1 is easily degraded, so it is not suitable for therapeutic use. A variety of long-acting GLP-1 analogs have been developed to treat obesity, DM and other related diseases [6]. Exendin-4 (Ex4), Liraglutide, Tirzepatide and Semaglutide are all powerful agonists of GLP-1, which have longer durations compared with GLP-1 effects, can reduce plasma glucose levels, slow down gastric emptying, and inhibit food intake [7-9], with common side effects including nausea, vomiting, headache, etc. [10-12]

### SUMMARY

The present disclosure provides a novel GLP-1 analog, which is derived from mutations of the human GLP-1 sequence at specific sites. Compared with the natural sequence of GLP-1, the novel GLP-1 analog can function more effectively in vivo, including but not limited to improving GLP-1 related diseases such as obesity, DM and FL. The novel GLP-1 analog can significantly improve the symptoms of mood disorders such as anxiety and depression.

To achieve the above objective, the present disclosure first provides GLP-1 analogs, and the GLP-1 analogs include the amino acid sequence of Formula I:

Formula I
(SEQ ID NO: 2)
HX₂X₃ GTF TSD VSS YLE GQA AKX₂₁ FIA WLX₂₇ KX₂₉RX₃₁

In the premise of not changing the secondary structure of Formula I, substitutions of the specific sites of the amino acids are achieved according to the principle of conservative substitution of amino acids, where,
X₂ is selected from a group consisting of valine, isoleucine, glycine, threonine, leucine and serine;
X₃ is selected from a group consisting of serine and alanine;
X₂₁ is selected from a group consisting of arginine and lysine;
X₂₇ is selected from a group consisting of serine and alanine;
X₂₉ is selected from a group consisting of alanine, serine, threonine and methionine; and
X₃₁ is selected from a group consisting of alanine, serine, threonine and methionine.

In some non-limiting embodiments, the GLP-1 analog includes a polypeptide sequence of Formula II:

Formula II
(SEQ ID NO: 1)
HGE GTF TSD VSS YLE GQA AKE FIA WLV KSRA

The second aspect of the present disclosure provides a composition, including the GLP-1 analog of Formula I or Formula II and a pharmaceutically acceptable excipient.

The third aspect of the present disclosure provides use of the GLP-1 analog or the composition in the treatment of GLP-1 related diseases; or use of the GLP-1 analog or the composition in the preparation of medicines for treating GLP-1 related diseases; or a method of treating GLP-1 related diseases, including administering a therapeutically effective amount of the GLP-1 analog or the composition to a subject in need.

In some non-limiting embodiments, the GLP-1 related diseases include obesity and related complications, diabetes mellitus and related complications, glucose intolerance and related diseases caused therefrom, hyperglycemia, hyperinsulinemia, hypertension, dyslipidemia, cognitive impairment, atherosclerosis, myocardial infarction, cardiovascular disease, stroke, related diseases caused by intestinal permeability disorders such as inflammatory bowel syndrome and/or dyspepsia and/or ulcerative colitis and/or Crohn's disease, apoplexy and/or hemorrhagic apoplexy, neuroinflammation, rheumatic or rheumatoid arthritis, kidney disease, asthma, chronic obstructive pulmonary disease, dysmetabolic syndrome, anxiety, depression, emotional disease and neurodegenerative disease of Alzheimer's disease, systemic lupus erythematosus, etc.

The present disclosure provides use of the GLP-1 analog or the composition in the preparation of medicines for reducing food intake and increasing insulin sensitivity.

The present disclosure provides use of the GLP-1 analog or the composition in the preparation of medicines for inhibiting fat accumulation.

In some non-limiting embodiments, the fat accumulation includes subcutaneous fat accumulation, inter-organ fat accumulation, and hepatic fat accumulation.

In some non-limiting embodiments, the present disclosure provides use of the GLP-1 analog or the composition in the preparation of medicines for treating anxiety and/or depression.

The present disclosure provides use of the GLP-1 analog or the composition in the preparation of health care products, nutritional supplements and as food additives.

Unless otherwise indicated, the quantities used to represent different components and reaction conditions herein can be interpreted as “approximately” or “about” in any case. Accordingly, unless otherwise specified, the numerical parameters quoted in the following description and claims are approximate parameters. Due to different standard errors under respective experimental conditions, different numerical parameters may be obtained.

### Beneficial Effects:

The present disclosure relates to a novel human glucagon-like peptide-1 (GLP-1) analog, which compared with the natural sequence of GLP-1, has a remarkable effect in improving GLP-1-related diseases such as obesity, type 2 diabetes mellitus (T2DM), and fatty liver (FL), as well as mood disorders such as anxiety and depression.

### BRIEF DESCRIPTION OF THE DRAWINGS

FIG. 1 illustrates the effect of a polypeptide of Formula II (referred to as G1 in the figure, the same below) on HEK-293t cell toxicity in a CCK-8 detection experiment.

FIG. 2 illustrates the effect of a polypeptide of Formula II on a cAMP content in a detection experiment of a cyclic adenosine monophosphate (AMP) kit.

FIGS. 3A and 3B illustrate the effect of a polypeptide of Formula II on the body weight and food intake of high-fat diet (HFD) mice, where FIG. 3A illustrates the effect of a polypeptide of Formula II on the body weight of HFD mice; FIG. 3B illustrates the effect of a polypeptide of Formula II on the food intake of HFD mice.

FIGS. 4A-D illustrate the effect of a polypeptide of Formula II on anxiety and depression symptoms in mice, where FIG. 4A shows the time of stays in the center area of an open field test;

FIG. 4B shows the number of stays in the center area of an open field test; FIG. 4C shows the time of stay in the open arm area of a plus maze test; and FIG. 4D shows the immobile time of a tail suspension test.

FIGS. 5A and 5B illustrate the effect of a polypeptide of Formula II on blood glucose of HFD mice in a glucose tolerance test (GTT) and an insulin tolerance test (ITT), where FIG. 5A illustrates the effect of a polypeptide of Formula II on blood glucose of HFD mice in a glucose tolerance test (GTT); FIG. 5B illustrates the effect of a polypeptide of Formula II on blood glucose of HFD mice in an insulin tolerance test (ITT).

FIGS. 6A and 6B illustrate the effect of a polypeptide of Formula II on fatty liver in an Oil Red O staining test, where FIG. 6A shows the Oil Red O staining of the liver, and FIG. 6B shows the quantification of the Oil Red O staining of the liver.

FIGS. 7A and 7B illustrate the effect of a polypeptide of Formula II on muscle in an hematoxylin eosin (HE) staining test, where FIG. 7A shows muscle HE staining, and FIG. 7B shows muscle HE quantification.

FIG. 8 illustrates a grouping diagram of mouse experiments in Example 5.

### DETAILED DESCRIPTION

The following description sets forth exemplary embodiments of the present disclosure. It should be appreciated that such description is not intended as a limitation to the scope of the present disclosure, but is provided as a description of exemplary embodiments.

The term “GLP-1 analog” used herein refers to analogs of human glucagon-like peptide-1 (GLP-1) or variants thereof, and is used to refer to polypeptides of Formula 1 and Formula II. The “GLP-1 analog” has GLP-1 activity, which refers to the ability to bind to GLP-1 receptors and trigger signal transduction pathways to produce insulinotropic effects or other physiological effects.

The term “amino acid” used herein includes compounds of the following general structure:

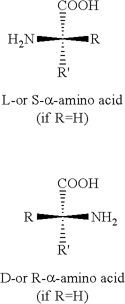

where R and R′ are as discussed herein, unless otherwise stated, used alone or as part of another group, the term “amino acid” includes an amino group attached to the same carbon designated as the “a” carbon and a carboxyl group, where R and/or R′ may be natural or unnatural side chains, including hydrogen. The absolute “S” configuration at the “a” carbon is often referred to as the “L” or “natural configuration”. In a case that both “R” and the “R substituent” are hydrogen, the amino acid is glycine and is not chiral. Unless otherwise stated, the term “amino acid” used herein alone or as part of another group includes, but is not limited to, natural or unnatural amino acids, and where the carboxyl group is replaced (reduced) with methanol such as valine, glycine, alanine, arylalanine, heteroarylalanine.

The GLP-1 analog of the present disclosure can be prepared by chemical synthesis using various solid-phase technologies^{[13-15]}.

The strategy used in the present disclosure is based on the temporarily protected Fmoc (9-fluorenylmethoxycarbonyl) group for the α-amino group, combined with the temporarily protected tert-butyl for the amino acid side chain^{[13,14,16]}.

The GLP-1 analog of the present disclosure can be synthesized stepwise starting from the C-terminal of the peptide on an insoluble polymeric carrier (also known as “resin”). Synthesis begins by attaching the C-terminal amino acid of the peptide to the resin via the formation of an amide or ester bond, which allows the obtained peptide to be ultimately released as the C-terminal amide or carboxylic acid, respectively. Alternatively, if a C-terminal amino acid is present, the C-terminal residue described herein can be linked to 2-methoxy-4-alkoxybenzyl alcohol resin, and after the completion of the assembly for the peptide sequence, the obtained peptide is released using a THF solution of LiBH₄ ^{[17].}

It is known to those skilled in the art that amino acids exist as two isomers, D and/or L, and the present disclosure includes a mixture of one isomer and/or another isomer of amino acids for use in the synthesis of peptides described herein.

The GLP-1 analog of the present disclosure can be produced by recombinant methods, that is, by culturing host cells that contain a DNA sequence encoding the analog and are capable of expressing the peptide in a suitable nutrient medium under conditions of allowing expression of the peptide. Non-limiting examples of host cells suitable for expression of these peptides are: Escherichia coli, Saccharomyces cerevisiae, and mammalian BHK or CHO cell lines. In some non-limiting embodiments, this fully recombinant fermentation step of the production process is desirable, for example for reasons of production economics. The fusion protein inclusion body containing the GLP-1 analog main chain is denatured and renatured to obtain a fusion protein containing the correct conformation. After a series of treatments such as enzyme digestion, sedimentation, and centrifugation, a higher content of GLP-1 analog main chain is obtained. Through ion exchange chromatography and purification, the GLP-1 analog main chain with a higher purity is obtained after treatment.

The term “excipient” broadly refers to any ingredient other than the active therapeutic ingredient. Excipients may be pharmaceutically acceptable inert substances, inactive substances and/or substances that are not pharmaceutically active. The formulation of active pharmaceutical ingredients with different excipients is known in the art [18]. Non-limiting functional examples of excipients include, but are not limited to, fillers, binders, lubricants, solvents, disintegrants, buffers, preservatives, and the like. Professional technicians in this field can easily select excipients with appropriate functions according to the dosage form of the formulation. For suitable carriers, please refer to literature

The term “filler” includes, but is not limited to, lactose, sucrose, etc.; the term “binder” includes but is not limited to starch, gelatin, etc.; the term “lubricant” includes but is not limited to magnesium stearate, etc.; the term “preservative” includes but is not limited to sodium benzoate, etc.; the term “disintegrant” includes but is not limited to methylcellulose, dry starch agar, etc.; the term “buffer” includes but is not limited to sodium chloride, etc.; and the term “solvent” includes but is not limited to ethanol, water, etc.

The term “GLP-1 related disease” refers to diseases related to GLP-1 receptor agonism, and/or capable of being improved by agonizing the GLP-1 receptor; these diseases include, but are not limited to, obesity, diabetes mellitus and related complications, glucose intolerance and related diseases caused therefrom, hyperglycemia, hyperinsulinemia, hypertension, dyslipidemia, cognitive impairment, atherosclerosis, myocardial infarction, cardiovascular disease, stroke, related diseases caused by intestinal permeability disorders, inflammatory bowel syndrome and/or dyspepsia and/or ulcerative colitis and/or Crohn's disease, apoplexy and/or hemorrhagic apoplexy, neuroinflammation, rheumatic or rheumatoid arthritis, kidney disease, asthma, chronic obstructive pulmonary disease, dysmetabolic syndrome, anxiety, depression, emotional diseases and neurodegenerative diseases of Alzheimer's disease, systemic lupus erythematosus, etc.

The scope of the present disclosure includes the GLP-1 analog and the composition containing the GLP-1 analog, where the composition includes the GLP-1 analog as the active ingredient and the pharmaceutically acceptable excipient that can be used in combination with GLP-1. The GLP-1 analog of the present disclosure is used alone or in combination with one or more therapeutic agents for other diseases to treat related diseases; and use in the preparation of medicines for reducing food intake, reducing β-cell apoptosis, increasing pancreatic islet β-cell function, and increasing β-cell mass and/or restoring glucose sensitivity to β-cells.

The GLP-1 analog of the present disclosure can be administered pharmaceutically in a variety of dosage forms (each dosage form includes immediate-release, sustained-release or sustained-controlled-release dosage forms), including but not limited to tablets, capsules, injections and other dosage forms. Those skilled in the art can select appropriate formulation forms according to the administration route. The administration route includes but is not limited to oral, transdermal, subcutaneous, intravenous, intramuscular, etc. The dosage form of the formulation is prepared according to the administration route and based on acceptable pharmaceutical operating procedures^{[20]}. All dosage forms used are well-known to those skilled in the pharmaceutical field, and pharmaceutically acceptable excipients are selected according to the selected administration route and standard pharmaceutical operating procedures and then administered together.

The GLP-1 analog or composition of the present disclosure can also be coupled with soluble polymers, such as targeted drug carriers, cross-linked with biodegradable polymers for controlling drug release and hydrogels, or amphiphilic block copolymers are combined for administration.

The dosage regimen of the formulations of the present disclosure will vary depending on known factors such as the pharmacokinetic properties of the particular agent and its mode and administration route, the species, health status, medical status, age, gender and body weight of the subject, as well the nature and extent of the symptoms, the type and frequency of treatment, the patient's renal and liver function, and the desired effect. The physician or veterinarian may prescribe the amount of medicine required to be effective in preventing, combating, or inhibiting the progression of the disease state.

Administration of therapeutic agents of the present disclosure includes, but is not limited to, administration of a therapeutically effective amount of the agent of the present disclosure. The term “therapeutically effective amount” refers to an amount of a therapeutic agent that treats or prevents a disease treatable by administration of a composition of the present disclosure. The dosage is an amount sufficient to show a therapeutic or preventive or ameliorative effect. The effects include treatment or prevention of the disease listed herein. The precise effective amount administered to the subject will depend on the subject's weight and state of health, the nature and extent of the disease to be treated, the recommendations of the treating physician, and the therapy or combination of therapies selected for administration. Therefore, it would be useless to specify the exact effective amount in advance.

According to general guideline principles, the active ingredient will generally be present in the composition in an amount from about 0.5% to 95%, based on the total weight of the composition. The compositions of the present disclosure may be administered in a single daily dose, or the total daily dose may be administered in two, three or four doses per day. The composition of the present disclosure may also be administered in long-acting formulations, which will allow the active ingredient to be slowly released over days/weeks/months as desired.

Abbreviations

| Abbrevi- | | Abbrevi- | | Abbrevi- | | Abbrevi- | |
|---|---|---|---|---|---|---|---|
| ation | Full name | ation | Full name | ation | Full name | ation | Full name |
| Fmoc | 9-Fluorenyl | Ile | Isoleucine | g | gram | GLP-1 | Human |
| | methoxycarbonyl | | | | | | glucagon-like |
| | | | | | | | peptide-1 |
| tBu | Tertiary butyl | Leu | Leucine | mg | milligram | DM | Diabetes |
| | | | | | | | mellitus |
| TFA | Trifluoroacetic | Lys | Lysine | rt or RT | Room | CVD | Cardiovascular |
| | acid | | | | temperature | | disease |
| OtBu | Tert-butoxy | Met | Methionine | sat. or | Saturated | AFLD | Alcoholic fatty |
| | | | | sat'd | | | liver |
| Pd/C | Palladium/ | Phe | Phenyl | aq. | Aquiferous | DPP-4 | Dipeptidyl |
| | carbon | | alanine | | | | peptidase 4 |
| Boc | Tert-butoxy | Pro | Proline | nm | Nanometer | GPCR | G-protein |
| | carbonyl | | | | | | coupled |
| | | | | | | | receptor |
| HOBT | 1-hydroxy | Ser | Serine | mL or | Millilitre | HFD | High-fat diet |
| | benzotriazole | | | ml | | | |
| MeCN | Acetonitrile | Thr | Threonine | mol | mole | ITT | Insulin |
| | | | | | | | tolerance test |
| Ala | Alanine | Trp | Tryptophan | mmol | mM | Sc or SC | Hypodermic |
| Arg | Arginine | Tyr | Tyrosine | Meq. | milliequivalent | GLP- | GLP-1 receptor |
| | | | | | | RA | agonists |
| Asn | Asparagine | Val | Valine | mp | Melting point | FL | Fatty liver |
| Asp | Aspartic acid | Dhval | 2,3-didehydro | PBS | Phosphate | IDF | International |
| | | | valine | | Buffered Saline | | diabetes |
| | | | | | | | federation |
| Cys | Cysteine | Dhthr | Dehydroxy | DCM | Dichloromethane | NAFLD | Nonalcoholic |
| | | | threonine | | | | fatty liver |
| Gln | Glutamine | Dah | 2,7-diamino | NMM | N- | NEP | Neutral |
| | | | heptanoic | | Methylmorpholine | | endopeptidase |
| | | | acid | | | | |
| Glu | Glutamic acid | Orn | Ornithine | DMF | Dimethylformamide | cAMP | Cyclic |
| | | | | | | | adenosine |
| | | | | | | | monophosphate |
| Gly | Glycine | Dhser | Dehydroxy | THF | Tetrahydrofuran | GTT | Glucose |
| | | | serine | | | | tolerance test |
| His | Histidine | Dap | 2,3-diamino | IP or ip | Intraperitoneal | / | / |
| | | | propionic | | | | |
| | | | acid | | | | |

### Example 1: General Method for Chemical Synthesis of Polypeptide of Formula I

Formula I
HX₂X₃ GTF TSD VSS YLE GQA AKX₂₁ FIA WLX₂₇ KX₂₉RX₃₁

In the premise of not changing the secondary structure of Formula I, substitutions of the specific sites of the amino acids were achieved according to the principle of conservative substitution of amino acids, where,
X₂ was selected from a group consisting of valine, isoleucine, glycine, threonine, leucine and serine;
X₃ was selected from a group consisting of serine and alanine;
X₂₁ was selected from a group consisting of arginine and lysine;
X₂₇ was selected from a group consisting of serine and alanine;
X₂₉ was selected from a group consisting of alanine, serine, threonine and methionine; and
X₃₁ was selected from a group consisting of alanine, serine, threonine and methionine.

Formula I of the present disclosure could be synthesized stepwise from the C-terminal of the peptide on an insoluble polymeric carrier (also known as “resin”). Synthesis begun by attaching the C-terminal amino acid of the peptide to the resin via the formation of an amide or ester bond, which allowed the obtained peptide to be ultimately released as the C-terminal amide or carboxylic acid, respectively. Alternatively, if a C-terminal amino acid was present, the C-terminal residue described herein could be linked to 2-methoxy-4-alkoxybenzyl alcohol resin (SASRIN™, BachemBioscience, Inc., King of Prussia, PA), and after the completion of the assembly for the peptide sequence, the obtained peptide was released using a THF solution of LiBH₄ ^{[17].}

### Example 2: General Method for Biosynthesis of Polypeptide (G1) of Formula II

a. Resin swelling: Fmoc-Ala-Wang resin was poured into a reaction column, to be soaked for 30 min by adding DCM, and drained.
b. Deprotection: an appropriate amount of deprotection solution was added to the reaction column, stirred with nitrogen for 30 min, and drained.
c. Weighing materials: a protected amino acid that was 3 times of a molar amount of the resin was weighed, and 3 times of the molar amount of benzotriazole-N,N,N,N-tetramethylurca hexafluorophosphate (HBTU) was weighed.
d. Deprotection washing: an appropriate amount of DMF was added to the reaction column, agitated with nitrogen for 2 min, drained, and the operation was repeated for 6 times.
e. Feeding: the prepared protected amino acid and HBTU were added to the reaction column, NMM that was 6 times of the molar amount of the resin was added, and stirred with nitrogen for 30 min.
f. Washing after reaction: the solution in the reaction column was drained, an appropriate amount of DMF was added for washing, the solution was agitated with nitrogen for 2 min, drained, and the operation was repeated for 3 times.
g. Detection: an appropriate amount (10 to 20 pieces) of resin was taken into a small test tube, and two drops each of solutions A, B, and C were added to be placed in a dry heater for being heated for 3 min (110° C.). If the solution was slightly yellow in color and the resin was colorless and transparent, the reaction was complete and the next amino acid could be connected. Steps b-f above were repeated until the last amino acid was connected. (Liquid A: 80% phenol+20% absolute ethanol; liquid B: redistilled pyridine; liquid C: 5 g ninhydrin+100 mL absolute ethanol).
h. Wash and dry after synthesis: after the last amino acid had been connected, deprotected and washed, it was drained, an appropriate amount of methanol was added to the reaction column, agitated with nitrogen for 2 min, and drained, then an appropriate amount of DCM was added, agitated with nitrogen for 2 min, and drained, and the operation was repeated for 3 times. An appropriate amount of methanol was added to a reaction kettle, agitated with nitrogen for 2 min, and drained, and the operation was repeated twice. The resin was put into a container and vacuum dried in a vacuum desiccator for 12 h.
i. Polypeptide cleavage: after being sealed, a centrifuge tube was put into a centrifuge to be centrifuged for 3 min at 4,000 rpm, a supernatant was removed, an ether was added to stir evenly, then centrifugation was performed again, and washing was repeated for 5 times. The polypeptide was put into a vacuum dryer to be dried for 24 h to obtain a crude white powder polypeptide, and the crude white powder polypeptide was weighed.

### Example 3: Cytotoxicity of Polypeptide of Formula II

HEK-293t cells were cultured in 96-well plates (37±0.5° C., 5±0.1% CO₂). 0.132 mg of polypeptide of Formula II was dissolved in 10 ml of PBS (4 μmol/mL), after the polypeptide was diluted according to the concentration gradient (2 μmol/mL, 1 μmol/mL, 0.5 μmol/mL, and 0.25 μmol/mL), a high-glucose DMEM complete medium (Wuhan Servicebio, the same below) was added, respectively. A CCK-8 kit (Wuhan Pricella Biotechnology) was used for toxicity detection, the absorbance at 450 nm was measured with a microplate reader and a cell survival rate was calculated, and an IC50 curve was drawn using GraphPad Prism (FIG. 1).

Based on the IC50 results of the polypeptide of Formula II, the dosage of administration of animals is estimated according to the following formula:

Dosage of administration of animals (mg/kg)=dosage of administration of cell (μg/mL)×liquid intake (mL/kg)/animal weight (kg)

The converted dosage of administration of animals is 1,134 μM/kg. In actual animal experiments, the dosage of administration of 60 μM/kg (approximately 5% of 1,134 μM/kg) was used.

### Example 4: Determination of Cyclic AMP

HEK-293t cells were cultured in 96-well plates, and a Lipo2000 transfection reagent (Beijing Solarbio Technology) was used to transfer the recombinant expression plasmid pCAGIG of the polypeptide of Formula II. After culturing for 24 h, a cAMP kit (Beijing Solarbio Technology) was used for detection, the absorbance at 450 nm was measured with a microplate reader, and GraphPad Prism was used for analysis.

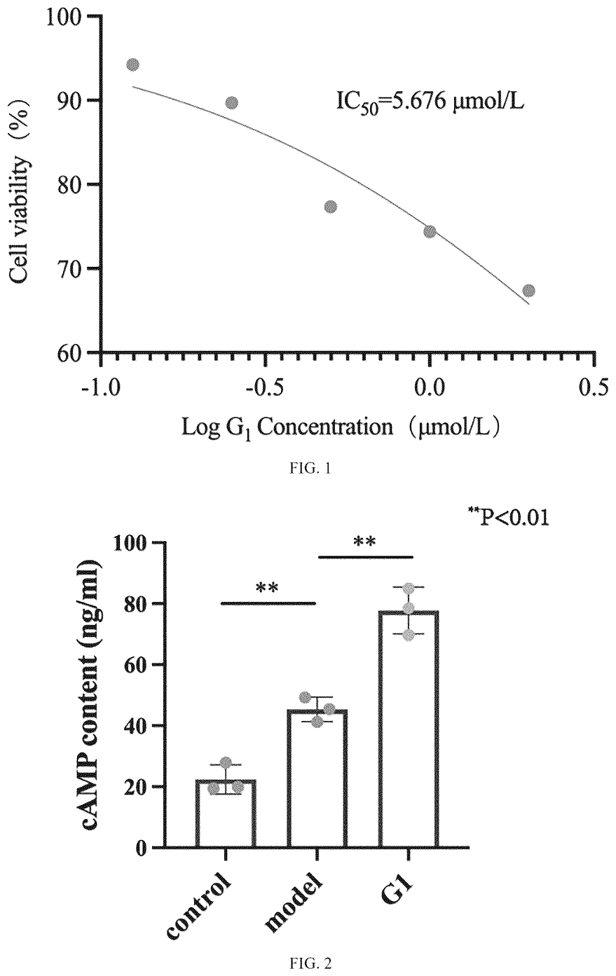

The results showed (as shown in FIG. 2) that the CAMP content in the polypeptide group of Formula II was significantly increased by about 30%-50% compared with a model group, indicating that the polypeptide of Formula II could activate its signaling pathway by binding to GPCR and increase intracellular second messenger concentration of cAMP through signal transduction, which was the same as the regulation of endogenous GLP-1. Therefore, the polypeptide of Formula II possessed the activity of endogenous GLP-1.

### Example 5: Effect of Polypeptide of Formula II on Body Weight

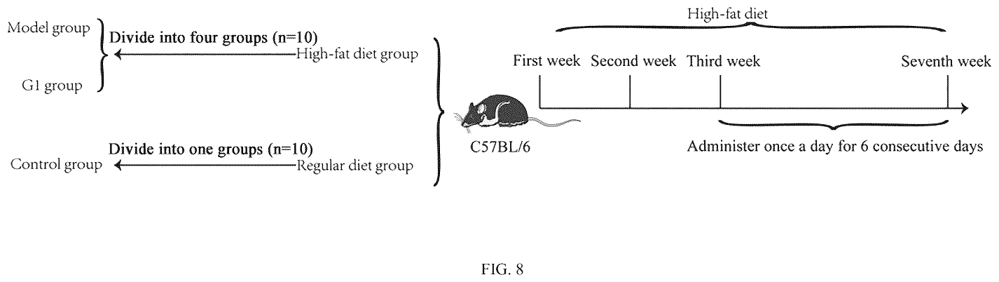

30 male C57BL/6 SPF grade mice (Beijing spfbiotech, the same below) were raised in an environment with a temperature of 22° C. to 26° C. and a relative humidity of 40% to 70%. The mice were divided into three groups (ten/group, as shown in FIG. 8): a normal control group (conventional feed and normal saline, NS), a HFD model group (high-fat feed and normal saline) and a Formula II polypeptide administration group (high-fat feed and polypeptide of Formula II, G1). On days 1-7, 50% high-fat feed was given for adaptive diet induction, and on days 8-50, 100% high-fat feed was given. On days 15-50, 0.9% normal saline or polypeptide of Formula II was injected subcutaneously every day, and daily body weight changes and total food intake 48 hours after each administration were measured.

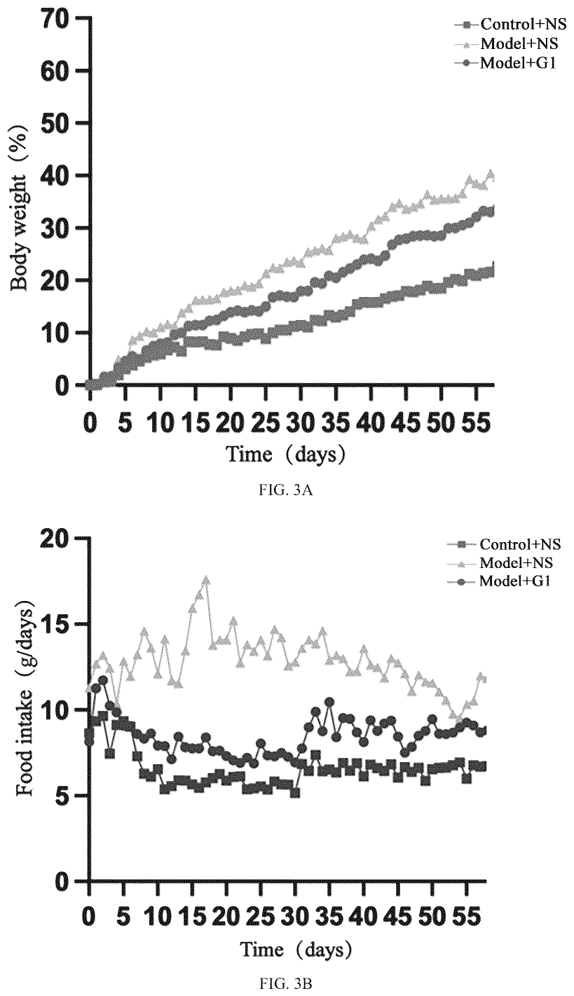

The body weight results were shown in FIG. 3A. Starting from the 20th day, the weight difference of the three groups of mice changed significantly. The normal group was given ordinary feed, and its weight increased slowly. Compared with the model group, the weight difference was about 10%-50%, and compared with the Formula II polypeptide group, the weight difference was about 5%-20%. Also given high-fat feed, the body weight of the model group increased by about 5%-20% compared with the Formula II polypeptide group.

The food intake results were shown in FIG. 3B. From days 6-50, the food intake of the model group increased by about 5%-80% compared with the normal control group. The food intake of the Formula II polypeptide group decreased by 20%-70% compared with the model group.

### Example 6: Anti-Anxiety and Depression Effects of Polypeptide of Formula II

Behavioral experiments were conducted on the three groups of mice in Example 5, respectively. Open Field Test (OFT) (Pullman): the mice were put into a test box and its activity during the test time was detected; elevated plus maze (EPM) (Pullman): the mice were placed in the center of the elevated plus maze and its activity during the test time was observed; and Tail suspension test (TST): the tails of the mice were suspended on a cage frame to observe their struggling, swinging and immobile behavior patterns while hanging.

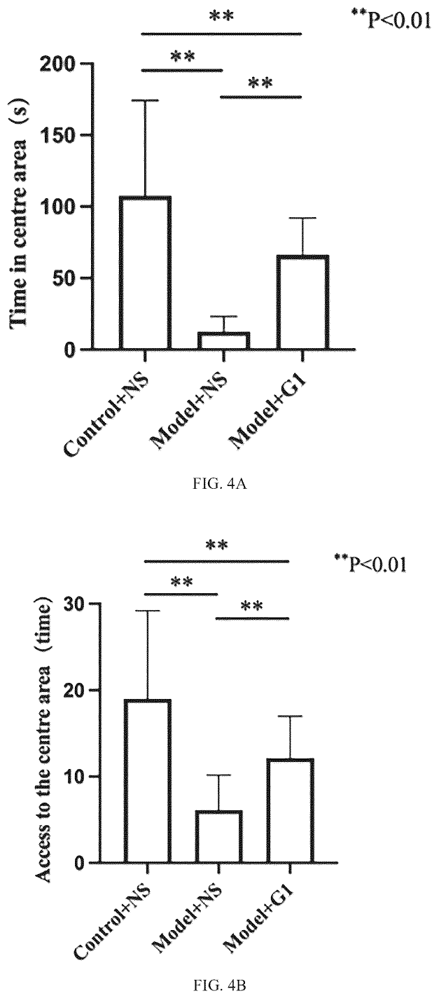

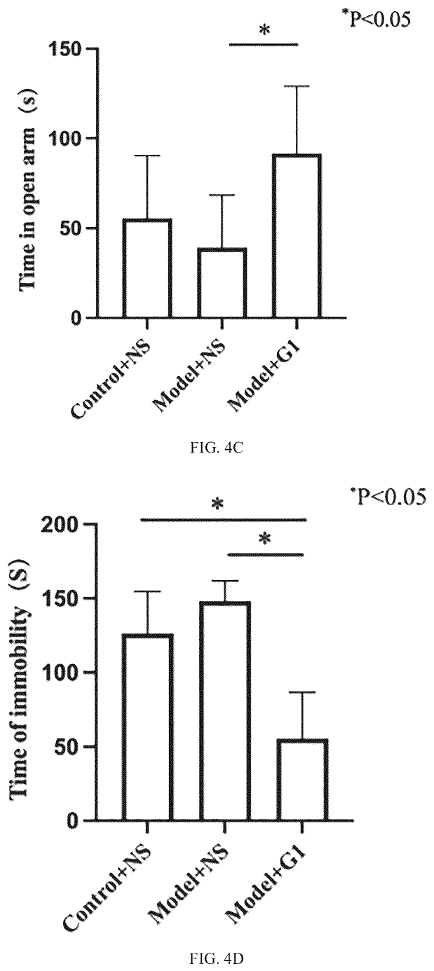

In OFT and EPM tests (as shown in FIG. 4A to FIG. 4C), compared with the control group, the entry time and number of mice in the model group were significantly reduced in the centre area, and the entry time in the open arm area was reduced; compared with the model group, the entry time and number of mice in the Formula II polypeptide administration group were significantly increased in the centre area, and the entry time in the open arm area was significantly increased. In the TST test (as shown in FIG. 4D), the immobility time of the mice in the model group was increased compared with the mice in the control group, and the immobility time of the mice in the Formula II polypeptide administration group was significantly lower than that of the mice in the model group and the control group.

The above results showed that obesity can aggravate the symptoms of anxiety and depression in mice. The polypeptide drugs of Formula II can significantly improve the symptoms of anxiety and depression in mice.

### Example 7: Effect of Polypeptide of Formula II on Blood Sugar

The mice in Example 5 were fasted for one day, during which they drank water normally. 20% glucose was administered intragastrically, and blood glucose values were measured using a blood glucose meter and blood glucose test paper at 15 min, 30 min, 60 min, 90 min, and 120 min.

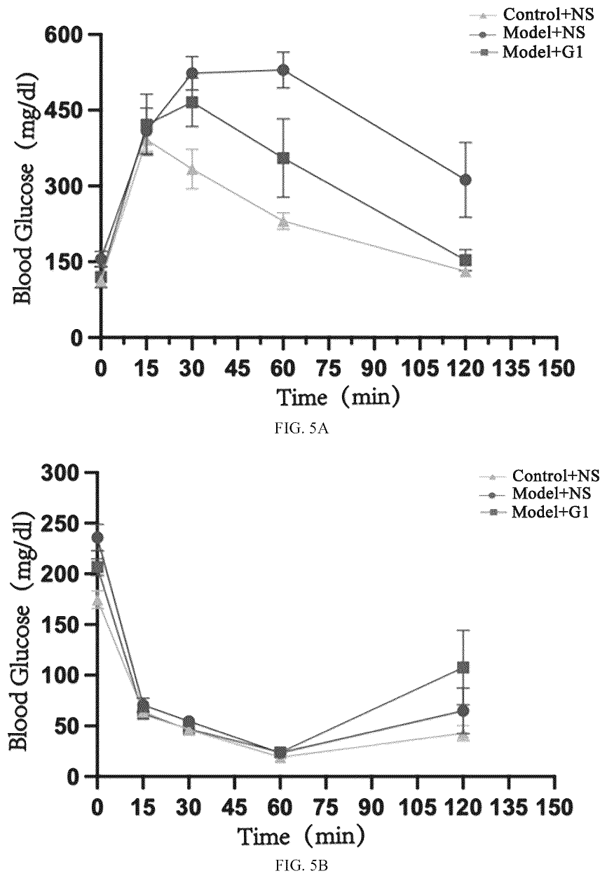

The GTT results were shown in FIG. 5A. The blood glucose of the mice in the model group increased significantly by 5%-35% within 15-120 min compared with the control group; and the blood sugar of the Formula II polypeptide administration group was 10%-35% lower than that of the model group within 30-120 min, indicating that the polypeptide of Formula II had an obvious effect on lowering blood sugar.

### Example 8: Effect of Polypeptide of Formula II on Insulin Resistance

The mice in Example 5 were fasted for one day, during which they drank water normally. 0.75 U/kg of insulin solution was injected intraperitoneally, and blood glucose values were measured with a blood glucose meter and blood glucose test paper at 15 min, 30 min, 60 min, 90 min, and 120 min.

The ITT results were shown in FIG. 5B. Within 120 min, the blood glucose of the mice in the control group decreased by 5%-60%, that of the mice in the model group decreased by 5%-40%, and that of the Formula II polypeptide administration group decreased by 10%-65%. Compared with the model group, the insulin resistance of the Formula II polypeptide administration group was improved.

### Example 9: Effect of Polypeptide of Formula II on Fat Accumulation in the Body

After the mice in Example 5 were anesthetized, they were fixed on a mouse board in a supine position, the outer skin was dissected, the subcutaneous fat was exposed, and the subcutaneous fat accumulation was recorded. The organs were then exposed and fat accumulation between the organs was observed. Finally, the mouse muscle and liver tissues were collected and stored in a-80° C. refrigerator.

The subcutaneous and inter-organ fat accumulation of mice in the model group increased by 30%-50% compared with the control group. The Formula II polypeptide administration increased by 20%-30% compared with the control group and decreased by 5%-25% compared with the model group, indicating that the Formula II polypeptide administration group had the obvious effect in reducing and controlling subcutaneous and inter-organ fat accumulation.

### Example 10: Effect of Polypeptide of Formula II on Fatty Liver

The mouse liver tissues collected in Example 9 were frozen and sectioned. Liver tissue sections were fixed with formaldehyde-calcium, washed, and immersed in 60% isopropyl alcohol. The saturated Oil Red O stock solution (Beijing Solarbio Technology) was diluted 3:2 (Oil Red O: distilled water). Sections were stained, and mounted with glycerin gelatin for microscopic examination.

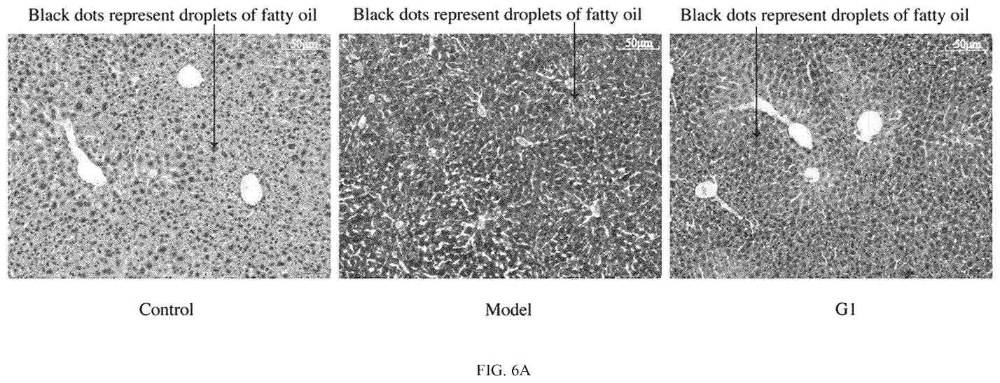

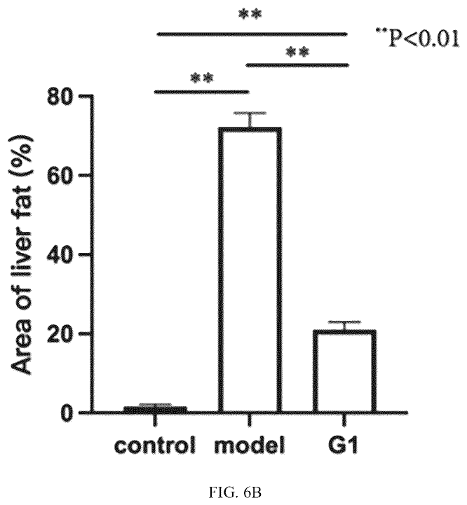

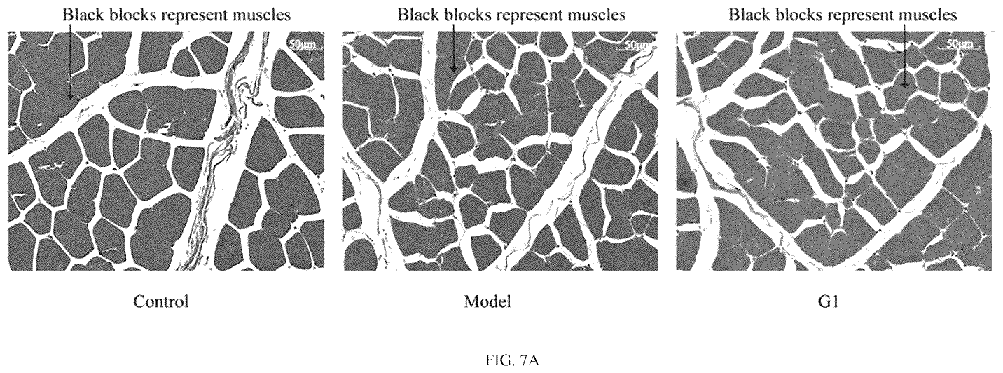

The results were shown in FIG. 6. The fat oil droplets in the model group increased by 40%-99% compared with the control group, and the fat oil droplets in the Formula II polypeptide administration group decreased by 20%-30% compared with the model group, indicating that the polypeptide of Formula II had the obvious effect in reducing liver fat accumulation.

### Example 11: Effect of Polypeptide of Formula II on Muscles

The mouse muscle tissues collected in Example 9 were frozen and sectioned, stained with hematoxylin and eosin (Beijing Solarbio Technology), mounted with glycerol gelatin for microscopic examination.

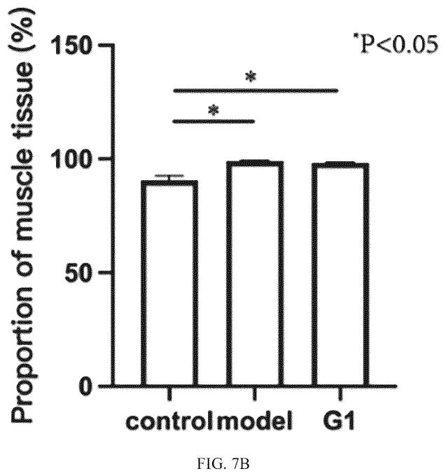

The results were shown in FIG. 7. The muscle area of the mice in the model group increased by 5%-15% compared with the control group, and there was no significant difference in the muscle area of the mice in the Formula II polypeptide administration group and the model group, indicating that the continuous administration of the polypeptide of Formula II did not affect the mice muscle content.

### Example 12: Preparation of Medicine Formulation of Polypeptide of Formula II

### A) Tablets

Tablets can be prepared according to conventional tablet preparation procedures to obtain tablets with the formulation specifications, for example, 100 mg of polypeptide of Formula II, 0.2 mg of colloidal silica, 5 mg of magnesium stearate, 275 mg of microcrystalline cellulose, 11 mg of starch, and 98.8 mg of lactose. Appropriate coatings can be used to improve taste or delay absorption and be prepared as 1,000 tablets.

### B) Capsules

Capsules can be prepared according to conventional capsule preparation procedures to obtain capsules of the formulation specifications. For example, 100 mg of polypeptide of Formula II, 150 mg of lactose, 50 mg of cellulose, 6 mg of magnesium stearate and an appropriate amount of absorption enhancer were filled into standard hard gelatin capsules and prepared as 1,000 capsules.

### C) Injections

Injections can be prepared through conventional procedures, such as preparing injection formulations with the following composition according to the method described below.

| Component | Amount |
|---|---|
| Polypeptide of Formula II | 5 mg |
| HCl or NaOH | Adjust pH to 6-8 |
| SBE-Cyclodextrin (Captisol) | 40 mg |
| Water for injection | Add to 1 L |
| Sodium chloride aqueous solution | Appropriate amount |

The weighed polypeptide of Formula II was dissolved in a portion of water for injection with an optimal pH value. SBE-cyclodextrin was added to the drug solution and stirred for 8 minutes approximately. NaOH or HCl was added to adjust the pH to between 6 and 8. Water for injection and a sodium chloride aqueous solution were added respectively to make the final volume 1 L and make it isotonic, then distributed into appropriate ampoules to prepare 1,000 injections. Before adjusting the pH value, other inactive components such as solvents and cosolvents, solubilizers, emulsifiers or thickeners, chelating agents, antioxidants and reducing agents, antimicrobial preservatives, buffers, swelling agents, protective agents, tension regulators and special additives can be added as needed. Injection formulations must be sterile, pyrogen-free, and free of particulate matter in the case of solutions. It can be prepared by stirring in a pharmaceutically acceptable buffer, which may or may not contain cosolvents or other excipients, and the solution was made isotonic and sterilized with pharmaceutical sodium chloride before use.

The non-limiting embodiments of the present disclosure have been described above with reference to the accompanying drawings, but the scope of claims of the present disclosure is not limited. Any modifications, equivalent substitutions and improvements made by those skilled in the art without departing from the scope and essence of the present disclosure shall be within the scope of the appended claims of the present disclosure.

### REFERENCES

[1] Trzaskalski N A, Vulesevic B, Nguyen M A, et al. Hepatocyte-derived DPP4 regulates portal GLP-1 bioactivity, modulates glucose production, and when absent influences NAFLD progression [J]. JCI Insight, 2023, 8(2).
[2] Bifari F, Manfrini R, Dei Cas M, et al. Multiple target tissue effects of GLP-1 analogues on non-alcoholic fatty liver disease (NAFLD) and non-alcoholic steatohepatitis (NASH) [J]. Pharmacol Res, 2018, 137:219-29.
[3] Loos R J F, Yeo G S H. The genetics of obesity: from discovery to biology [J]. Nat Rev Genet, 2022, 23(2): 120-33.
[4] Galicia-Garcia U, Benito-Vicente A, Jebari S, et al. Pathophysiology of Type 2 Diabetes Mellitus [J]. International Journal of Molecular Sciences, 2020, 21(17): 6275.
[5] Stefan N, Schick F, Birkenfeld A L, et al. The role of hepatokines in NAFLD [J]. Cell Metab, 2023, 35(2): 236-52.
[6] Lafferty R A, Flatt P R, Irwin N. GLP-1/GIP analogs: potential impact in the landscape of obesity pharmacotherapy [J]. Expert Opin Pharmacother, 2023, 24(5): 587-97.
[7] Goldenberg R M, Steen O. Semaglutide: Review and Place in Therapy for Adults With Type 2 Diabetes [J]. Can J Diabetes, 2019, 43(2): 136-45.
[8] van Can J, Sloth B, Jensen C B, et al. Effects of the once-daily GLP-1 analog liraglutide on gastric emptying, glycemic parameters, appetite and energy metabolism in obese, non-diabetic adults [J]. Int J Obes (Lond), 2014, 38(6): 784-93.
[9] Yap M K, Misuan N. Exendin-4 from Heloderma suspectum venom: From discovery to its latest application as type II diabetes combatant [J]. Basic Clin Pharmacol Toxicol, 2019, 124(5): 513-27.
[10] Madsbad S. Exenatide and liraglutide: different approaches to develop GLP-1 receptor agonists (incretin mimetics)—preclinical and clinical results [J]. Best Pract Res Clin Endocrinol Metab, 2009, 23(4): 463-77.
[11] Rosenstock J, Wysham C, Frias J P, et al. Efficacy and safety of a novel dual GIP and GLP-1 receptor agonist tirzepatide in patients with type 2 diabetes (SURPASS-1): a double-blind, randomized, phase 3 trial [J]. Lancet, 2021, 398(10295): 143-55.
[12] Sorli C, Harashima S I, Tsoukas G M, et al. Efficacy and safety of once-weekly semaglutide monotherapy versus placebo in patients with type 2 diabetes (SUSTAIN 1): a double-blind, randomized, placebo-controlled, parallel-group, multinational, multicentre phase 3a trial [J]. Lancet Diabetes Endocrinol, 2017, 5 (4): 251-60.
[13] Gross E. The Peptides Analysis, Synthesis, Biology: Modern Techniques of Conformational Structural, and Configurational Analysis [M]. Elsevier, 2012.
[14] Schröder E, Lübke K. The Peptides: Methods of peptide synthesis [M]. Elsevier, 2014.
[15] Chan W, White P. Fmoc solid phase peptide synthesis: a practical approach [M]. OUP Oxford, 1999.
[16] Atherton E, Sheppard R. The fluorenylmethoxycarbonyl amino protecting group [J]. ChemInform, 1988, 19 (33): no-no.
[17] Bravis V, Kaur A, Walkey H C, et al. Relationship between islet autoantibody status and the clinical characteristics of children and adults with incident type 1 diabetes in a UK cohort [J]. BMJ Open, 2018, 8(4): e020904.
[18] Allen L V, Jr. Remington: The Science and Practice of Pharmacy: from the past into the future [J]. Int J Pharm Compd, 2012, 16(5): 358-62.
[19] Fox L M. The science and practice of pharmacy [J]. American Journal of Pharmaceutical Education, 2006, 70(3).
[20] Goodman L S. Goodman and Gilman's the pharmacological basis of therapeutics [M]. McGraw-Hill New York. 1996.

What is claimed is:

1. A glucagon-like peptide-1 (GLP-1) analog, comprising amino acid sequences of Formula I:
Formula I
(SEQ ID NO: 2)
HX₂X₃ GTF TSD VSS YLE GQA AKX₂₁ FIA WLX₂₇ KX₂₉RX₃₁
wherein,
X₂ is selected from the group consisting of valine, isoleucine, glycine, threonine, leucine and serine;
X₃ is selected from the group consisting of serine and alanine;
X₂₁ is selected from the group consisting of arginine and lysine;
X₂₇ is selected from the group consisting of serine and alanine;
X₂₉ is selected from the group consisting of alanine, serine, threonine and methionine; and
X₃₁ is selected from the group consisting of alanine, serine, threonine and methionine.

2. A method of treating GLP-1 related diseases, comprising administering a therapeutically effective amount of the GLP-1 analog according to claim 1 to a subject in need thereof.

3. The method according to claim 2, the GLP-1 related diseases comprise at least one of obesity and related complications, diabetes mellitus and related complications, glucose intolerance and related diseases caused therefrom, hyperglycemia, hyperinsulinemia, hypertension, dyslipidemia, cognitive impairment, atherosclerosis, myocardial infarction, cardiovascular disease, stroke, related diseases caused by intestinal permeability disorders such as inflammatory bowel syndrome and/or dyspepsia and/or ulcerative colitis and/or Crohn's disease, apoplexy and/or hemorrhagic apoplexy, neuroinflammation, rheumatic or rheumatoid arthritis, kidney disease, asthma, chronic obstructive pulmonary disease, dysmetabolic syndrome, anxiety, depression, mood disorders and neurodegenerative diseases of Alzheimer's disease, and systemic lupus erythematosus.

4. A method of reducing food intake, comprising administering a therapeutically effective amount of the GLP-1 analog according to claim 1 to a subject in need thereof.

5. A method of increasing insulin sensitivity, comprising administering a therapeutically effective amount of the GLP-1 analog according to claim 1 to a subject in need thereof.

6. A method of inhibiting fat accumulation, comprising administering a therapeutically effective amount of the GLP-1 analog according to claim 1 to a subject in need thereof.

7. The method according to claim 6, wherein the fat accumulation comprises subcutaneous fat accumulation, inter-organ fat accumulation, and hepatic fat accumulation.

8. A composition, comprising the GLP-1 analog according to claim 1 and a pharmaceutically acceptable excipient.

9. A food additive, comprising the composition according to claim 8.

10. A food additive, comprising the GLP-1 analog according to claim 1.

11. A GLP-1 analog, comprising a polypeptide sequence of Formula II:
Formula II
(SEQ ID NO: 1)
HGE GTF TSD VSS YLE GQA AKE FIA WLV KSRA

12. A method of treating GLP-1 related diseases, comprising administering a therapeutically effective amount of the GLP-1 analog according to claim 11 to a subject in need thereof.

13. The method according to claim 12, the GLP-1 related diseases comprise at least one of obesity and related complications, diabetes mellitus and related complications, glucose intolerance and related diseases caused therefrom, hyperglycemia, hyperinsulinemia, hypertension, dyslipidemia, cognitive impairment, atherosclerosis, myocardial infarction, cardiovascular disease, stroke, related diseases caused by intestinal permeability disorders such as inflammatory bowel syndrome and/or dyspepsia and/or ulcerative colitis and/or Crohn's disease, apoplexy and/or hemorrhagic apoplexy, neuroinflammation, rheumatic or rheumatoid arthritis, kidney disease, asthma, chronic obstructive pulmonary disease, dysmetabolic syndrome, anxiety, depression, mood disorders and neurodegenerative diseases of Alzheimer's disease, and systemic lupus erythematosus.

14. A method of reducing food intake, comprising administering a therapeutically effective amount of the GLP-1 analog according to claim 11 to a subject in need thereof.

15. A method of increasing insulin sensitivity, comprising administering a therapeutically effective amount of the GLP-1 analog according to claim 11 to a subject in need thereof.

16. A method of inhibiting fat accumulation, comprising administering a therapeutically effective amount of the GLP-1 analog according to claim 11 to a subject in need thereof.

17. The method according to claim 16, wherein the fat accumulation comprises subcutaneous fat accumulation, inter-organ fat accumulation, and hepatic fat accumulation.

18. A composition, comprising the GLP-1 analog according to claim 11 and a pharmaceutically acceptable excipient.

19. A food additive, comprising the composition according to claim 18.

20. A food additive, comprising the GLP-1 analog according to claim 11.


SEQUENCE LISTING (2 sequences)

SEQ ID NO: 1 - protein, 31 aa, synthetic construct
HGEGTFTSDVSSYLEGQAAKEFIAWLVKSRA

SEQ ID NO: 2 - protein, 31 aa, synthetic construct
  VAR_SEQ 2: note = valine, isoleucine, glycine, threonine, leucine, or serine
  VAR_SEQ 3: note = serine or alanine
  VAR_SEQ 21: note = arginine or lysine
  VAR_SEQ 27: note = serine or alanine
  VAR_SEQ 29: note = alanine, serine, threonine, or methionine
  VAR_SEQ 31: note = alanine, serine, threonine, or methionine
HXXGTFTSDVSSYLEGQAAKXFIAWLXKXRX